(19) United States
(12) Patent Application Publication
(10) Pub. No.: US 2022/0267601 A1
(43) Pub. Date: Aug. 25, 2022

(54) BIOPOLYMER AND METHOD OF PREPARING THE SAME

(71) Applicant: INVOVIRON INDUSTRIES TRADING PVT LTD., Shyam Nagar, Kanpur (IN)

(72) Inventors: Sarthak Gupta, Kakadev (IN); Shahid Jamal, Shyam Nagar (IN)

(21) Appl. No.: 17/620,569

(22) PCT Filed: Jun. 17, 2020
(86) PCT No.: PCT/IN2020/050534

§ 371 Date: Dec. 17, 2021

(30) Foreign Application Priority Data
Jun. 19, 2019 (IN) 201911024333

(51) Int. Cl. C08L 89/04; C08J 5/18; C08J 3/12

(52) U.S. Cl. CPC C08L 89/04; C08J 5/18; C08J 3/12; C08L 2201/06; C08L 2203/16; C08L 2203/30; C08J 2389/04

(57) ABSTRACT

The present invention relates to a method of making biopolymer including but not limited to bio plastic from animal by-products, more specifically from poultry feathers wherein the method of making a biopolymer comprises the steps: i) i) pre-treatment of native feathers; ii) extraction of keratin protein from pre-treated feathers in the presence of reducing agent; iii) polymerization by blending keratin protein with one or more plasticizer, one or more additive and one or more cross-linking agent, optionally in presence of at least one alkali hydroxide to obtain a polymer compound using one or more thermal processing techniques at a temperature in the range of 60° C. to 150° C.; and iv) applying pressure and subjecting the polymer compound to thermal processing at a temperature in the range of 100° C. to 220° C. in presence of at least one or more excipients.

### FIELD OF THE INVENTION

The present invention relates generally to a method of preparing biopolymer from animal by-products and more specifically to a method of preparing bio plastic from feathers.

### BACKGROUND OF THE INVENTION

Biopolymers are polymers that can be found in or manufactured by living organisms or obtained from renewable resources that can be used to manufacture bio-plastics. Biopolymers can be divided also into two broad groups, namely biodegradable and non-biodegradable biopolymers. Depending on the specific application and some limitations, the biopolymers are often blended with one another or with synthetic polymers to improve total degradation time as well as mechanical properties or to reduce the cost of production. Table 1 below shows the list of existing biopolymers and raw material used along with their limitations:

| | | Raw | |
|---|---|---|---|
| Biopolymer | Feedstock | Material | Limitations |
| Starch Based | Corn, potato, | Starch | Low water vapour barrier |
| | wheat, tapioca | | Poor mechanical properties |
| | | | Bad processability |
| | | | Brittleness |
| Cellulose | Wood Pulp | Cellulose | Low water vapour barrier |
| Based | | | Poor mechanical properties |
| | | | Bad processability |
| | | | Brittleness |
| TPS | Potato, Corn | Starch | Sensitivity to moisture |
| | | | Retrogradation processes. |
| PHA & PHB | Bacterial | Bacillus | PHAs - ranging from stiff, |
| | fermentation | subtilis | brittle to semi rubber- |
| | | | like |
| | | | PHB has better oxygen |
| | | | barrier properties than |
| | | | both PP and PET, |
| | | | properties than PP, and |
| | | | fat and odour barrier |
| | | | properties that are |
| | | | sufficient for use in |
| | | | food packaging. |
| PLA | Corn (Major), | Lactic | Brittleness and low |
| | sugar beet, | Acid | crystallinity result into |
| | potatoes, wheat, | | low thermal stability and |
| | maize, tapioca | | limited applications. |
| PA 11 | Natural oil | 11- | Low Density, Low melting |
| | | aminound | point and glass transition |
| | | ecanoic | temperature |
| | | acid | |
| | | (Amine & | |
| | | Fatty | |
| | | acids) | |
| Chitosan | Fish | Chitin | Moderate tensile strength |
| | | | and modulus |
| | | | Low Heat tolerance |
| Keratin | Chicken | Keratin | Moderate tensile strength |
| Based | Feathers | | and modulus |
| | | | High water permeability |

Bio-plastics are created using biodegradable biopolymers which closely resemble synthetic plastic in terms of strength, robustness and durability. In the past, the researches were directed towards making bio-plastics from plant-based feedstock such as corn, wheat, sugarcane, potato, sugar beet, rice, bagasse, corn stover, wheat straw etc. Although these options are “eco-friendly” but the major drawback is associated with considerably higher cost of production, limited applications and lack of availability of raw material.

Feathers mainly contains keratin protein (˜90%) which can be extracted, processed and used into different forms for biotechnological application, such as sponges, films, fibers, alone or blended with other natural or synthetic polymers. Poultry feathers are easily available waste products of the poultry industry. Various studies have revealed that keratin proteins have been processed to form biopolymers using various techniques.

Indian patent application IN2688/KOLNP/2006 discloses a composition suitable for making films, wherein the composition contains keratin obtained from avian feathers and at least one OH containing plasticizer. The cited reference uses casting techniques to manufacture biodegradable polymer and fails to mention the steps of the present invention, in particular, use of chain extension mechanism to produce biodegradable polymer compound.

All the prior studies have suggested the casting technique for preparing biopolymer using keratin, and because of the same the overall cost of the product is very high, thus, making the technology industrially non-feasible.

Therefore, in light of the discussion above, there is a need to develop a biopolymer having improved mechanical and physical properties which may be used along with other natural polymers in a more efficient and inexpensive way for preparing such biopolymer including, but not limited to, bio plastics.

### OBJECT OF THE INVENTION

An aspect of the present invention provides an efficient and economically feasible method of preparing biopolymer including but not limited to bio plastic from animal by-products, in particular, poultry feather.

Another aspect of the present invention provides a biopolymer including, but not limited to, bio plastic prepared from the said method.

In yet another aspect of the present invention includes said biopolymer with any of the existing natural polymer or chemically synthesized polymer or microbial polyester, or a mixture thereof.

### SUMMARY OF THE INVENTION

The present invention is described hereinafter by various embodiments. This invention may, however, be embodied in many different forms and should not be construed as limited to the embodiment set forth herein. Rather, the embodiment is provided so that this disclosure will be thorough and complete and will fully convey the scope of the invention to those skilled in the art.

Embodiments of the present invention relate to a method of making biopolymer including, but not limited to, bio plastic from animal by-products, more specifically from feathers wherein the method of making a biopolymer comprises the steps:

i) pre-treatment of native feathers;

ii) extraction of keratin protein from pre-treated feathers in the presence of reducing agent;

iii) polymerization by blending keratin protein with one or more plasticizer, one or more additive and one or more cross-linking agent, optionally in presence of at least one alkali hydroxide, to obtain a polymer compound using one or more thermal processing techniques at a temperature in the range of 60° C. to 150° C.; and

iv) applying pressure and subjecting the polymer compound to thermal processing at a temperature in the range of 100° C. to 220° C. in presence of at least one or more excipients.

Another embodiment of the present invention aims to provide a biopolymer as obtained from the above method. The said biopolymer may be combined with any of the existing natural polymer, chemically synthesized polymer, microbial polyester or a mixture thereof.

### BRIEF DESCRIPTION OF DRAWINGS

So that the manner in which the above recited features of the present invention can be understood in detail, a more particular description of the invention, briefly summarized above, may have been referred by examples, some of which are illustrated in the appended drawings. It is to be noted, however, that the appended drawings illustrate only typical examples of this invention and are therefore not to be considered limiting of its scope, for the invention may admit to other equally effective examples.

These and other features, benefits, and advantages of the present invention will become apparent by reference to the following text figure, with like reference numbers referring to like structures across the views, wherein:

FIG. 1A illustrates that cleaned and defatted feathers are weighed and a solution containing sulphide ions is prepared.

FIG. 1B illustrates that cleaned and defatted feathers are dissolved in a solution containing sulphide ions at an elevated temperature with the help of overhead stirrer.

FIG. 1C illustrates that the solution is then filtered to obtain a solution containing dissolved ingredients.

FIG. 2A illustrates the step where keratin particles are precipitated out from the solution by adjusting the pH.

FIG. 2B illustrates the microporous keratin particles obtained after following the extraction step.

FIG. 2C illustrates that the extracted keratin is dissolved in solution containing hydroxide ions and is mixed with a solution containing plasticizers and additives. The mixture is then dried in an oven to obtain a film or membrane.

FIG. 3A illustrates an alternate method which is performed by mixing dry keratin with plasticizers, cross-linking agents and additives.

FIG. 3B illustrates that the mixture made above is subjected to external heating and pressure to obtain bio plastic strands.

FIG. 4A illustrates an article made using compression molding.

FIG. 4B illustrates the dumbbell shaped article produced using injection molding.

### DETAILED DESCRIPTION OF THE INVENTION

Various modifications to these embodiments are apparent to those skilled in the art from the description and the accompanying drawings. The principles associated with the various embodiments described herein may be applied to other embodiments. Therefore, the description is not intended to be limited to the embodiments shown with the accompanying drawings but is to provide broadest scope consistent with the principles and the novel and inventive features disclosed or suggested herein. Accordingly, the invention is anticipated to hold on to all other alternatives, modifications, and variations that fall within the scope of the present invention.

Keratin protein is insoluble in the majority of solvents, resistant to proteolytic enzymes, durable, chemically unreactive, and suitable to exposure to severe environmental conditions. Most of the outer layers of animal such as feather, hair, nail etc. comprises of keratin. Poultry feathers are a rich source of this tough protein called as keratin. Since feathers constitute an enormous portion of waste product in the poultry industry, their disposal poses a major challenge like that of soil pollution and thus efficient extraction is required. The poultry feathers collected from a farm or meat processing unit may be processed for forming a biopolymer, preferably bio-plastic.

In a preferred embodiment, the present invention relates to a method of making biopolymer from feathers comprising the steps of

i) pre-treatment of native feathers;

ii) extraction of keratin protein from pre-treated feathers in the presence of reducing agent;

iii) polymerization by blending keratin protein with one or more plasticizer, one or more additive and one or more cross-linking agent, optionally in presence of at least one alkali hydroxide, to obtain a polymer compound using one or more thermal processing techniques, at a temperature in the range of 60° C. to 150° C.; and

iv) applying pressure and subjecting the polymer compound to thermal processing at a temperature in the range of 100° C. to 220° C. in presence of at least one or more excipients.

In an embodiment, the starting material of the present invention may be any keratin containing animal by-products. Examples of keratin containing animal by-products include, but are not limited to, hair, nail, epidermis, hoof, horn, and feather. Preferably, poultry feathers in its native form are used as the starting material. As used herein, the term “poultry feather” includes, but is not limited to, chickens, turkeys, quails, ducks, geese, pigeons, doves, pheasants, emu, swans, and ostriches. That being said, although chicken feather is used as a poultry feather in some example of making the biopolymer, in other examples, the method of making the said biopolymer may use other types of poultry feathers, and the disclosure is not limited in this respect. In the present invention, the keratin protein may be preferably obtained from a group comprising feather fiber keratin, feather quill keratin, and mixtures thereof.

Although, the biopolymers may be prepared using a variety of different methods, in some examples, the method of the present invention comprises the first step of pre-treatment of poultry feathers which may be carried out in order to remove any extraneous materials from the feathers obtained from the poultry farms. Such a pre-treatment step may comprise an initial step of washing with water followed by exposure to various chemicals like Sodium dodecyl sulphate (SDS), Cetyl trimethyl ammonium bromide (CTAB), detergent, petroleum ether, acetone, and mixtures thereof. However, other conventional pre-treatments are also possible, provided they do not essentially change the material-characteristic structure of the feather. Further, the cleaned and defatted feathers are pulverized into powdered form using a pulveriser. Suitable amount of keratin protein may be required preferably in the range of 0 wt % to 90% wt %, more preferably between 30 wt % to 60 wt % of total weight of the biopolymer synthesised.

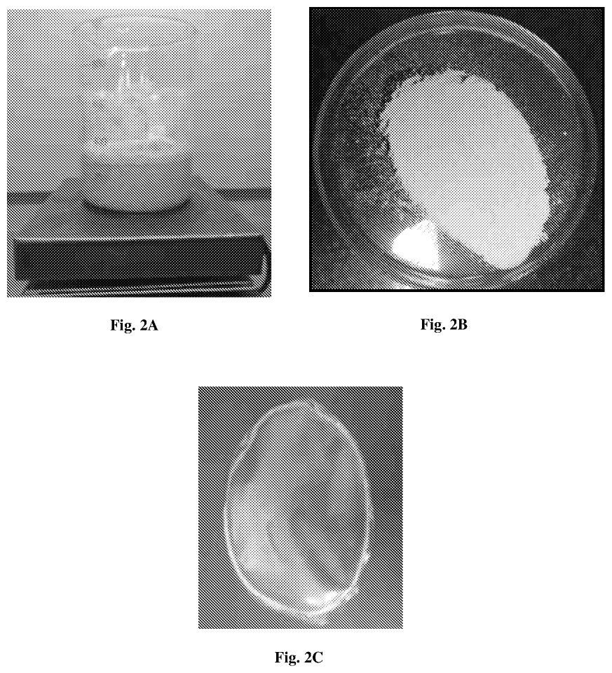

In another embodiment, after the pre-treatment step, the extraction of feather keratin from defatted feather may be performed using any suitable hydrolytic and reduction processes. Keratin may be extracted using reducing agents selected from a group comprising potassium cyanide thioglycolic acid, sodium sulphide, 2-mercaptoethanol, dithiothreitol, sodium m-bisulphite, and sodium bisulphite, sodium hydroxide, urea, thiourea, and mixtures thereof. The amount of reducing agent may be used in the range of 1 wt %-80 wt % of total weight of dried feathers. Basically, the reducing agents aids in increasing the solubility of the protein and to obtain microporous keratin particles as illustrated in FIG. 2B.

In another embodiment, the soluble keratin particles may be further processed with chemical agents that make the keratin molecules join together to form long chains, a process called polymerization. The chemical agents include, but are not limited to plasticizers, additives, cross-linking agents, alkali hydroxides and mixtures thereof. In the present invention, polymerization may be performed by blending keratin protein with one or more plasticizer, one or more additives, and one or more cross-linking agents, optionally, in the presence of at least one alkali hydroxide at a temperature in the range of 60 to 150° C. to obtain a mixture. The solution undergoes heat treatment and dried in an oven to create a polymer compound in the form of a film or membrane. The incorporation of additives may result into decrease in setting or drying time in the oven.

In an alternate embodiment, the keratin particles may be further processed with chemical agents at elevated temperatures that make the keratin molecules join together to form long chains using the chain extension mechanism. The chemical agents include, but are not limited to plasticizers, additives, cross-linking agents, alkali hydroxides and mixtures thereof. In the present invention, polymerization may be performed by blending keratin protein with one or more plasticizer, one or more additives, and one or more cross-linking agents at a temperature preferably in the range of 60° C. to 150° C., with or without the presence of at least one alkali hydroxide to obtain a compound polymer. Some external pressure may be optionally applied using a hammer.

Plasticizers are small, relatively non-volatile, organic molecules that are added to polymers to reduce brittleness, impart flexibility, and improve toughness, reducing crystallinity, lowering glass transition and melting temperatures. Suitable plasticizer that may be included, but are not limited to glycerol, sorbitol, ethanolamine, formamide, ethylene bisformide, glycerol triacetate, dibutyl tartrate and mixtures thereof. The amount of plasticizer required for making a biopolymer may range between 0 wt % to 30 wt % of the total weight of the biopolymer synthesised.

Additives may be incorporated for improving toughness and stability of biopolymer. Additives may be selected from the group comprising polymers, rayon, cellulose and mixtures thereof. Preferably, the additive that may be included, but are not limited to poly(lactic acid) [PLA], polyhydroxyalkanoate (PHA), polyhydroxybutyrate (PHB), polybutylene adipate terephthalate (PBAT), Polycaprolactone (PCL), Polybutylene succinate (PBS), Biodegradable Polypropylene (PP), Thermoplastic starch (TPS), Polyamide 11(PA11), Polyvinyl alcohol (PVA), Lyocell Fiber, and mixtures thereof. The amount of additives required for making a biopolymer may range between 0 wt % to 60 wt % of total weight of the biopolymer synthesised.

Cross-linking agents may be incorporated to induce crosslinking between various components for making the biopolymer. Suitable cross-linking agent that may be included, but are not limited to carboxymethyl cellulose, micro crystalline cellulose, starch, citric acid, Joncryl®, glutaraldehyde, propionic acid, lignin with glycine and mixtures thereof. The amount of cross-linking agents required for making a biopolymer may range between 0 wt % to 15 wt % of the total weight of the biopolymer synthesised.

Alkali hydroxides may be incorporated for shortening the chain length of the keratin molecules, both strongly alkaline inorganic substances and strong inorganic acids may be used. Preferably, strong alkaline inorganic substances such as alkali hydroxides and alkaline earth hydroxides may be used. More preferably, alkali hydroxides may be used, selected from a group comprising Lithium hydroxide (LiOH), Sodium hydroxide (NaOH), Potassium hydroxide (KOH), Rubidium hydroxide (RbOH), Caesium hydroxide (CsOH) and mixtures thereof. The amount of alkali hydroxides required for making a biopolymer may range between 0 wt % to 8wt % of the total weight of the biopolymer synthesised.

In an embodiment, the amount of plasticizer may be present in the range of 0 wt % to 30 wt %, additive may be present in the range of 0 wt % to 60 wt %, cross-linking agent may be present in the range of 0 wt % to 15 wt % and alkali hydroxide may be present in the range of 0 wt % to 8wt % of the total weight of the biopolymer synthesised.

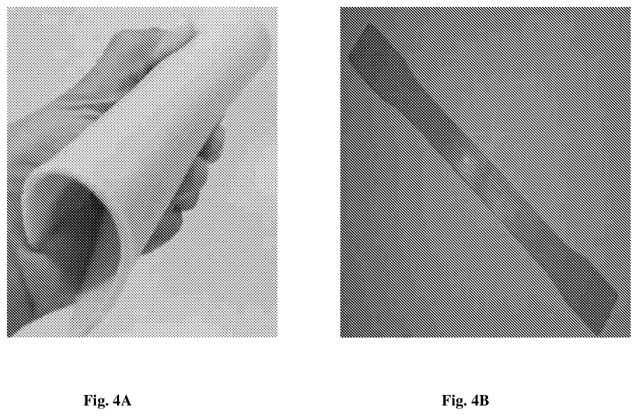

In another embodiment, the polymer compound obtained from the polymerization step may be exposed to external pressure and thermally processed with at least one or more excipients at a temperature in the range of 100° C. to 220° C. In an embodiment, thermal processing may be performed using one or more steps of kneading, extrusion molding, injection molding, blow molding, compression molding, transfer molding, thermoforming, casting, calendering, low-pressure molding, high-pressure laminating, reaction injection molding, foam molding, or coating to obtain biopolymer as the final product. Such conventional methods are suitably optimized and used in the present invention for obtaining biopolymer in various forms and shapes as illustrated in FIG. 4A and FIG. 4B. It is pertinent to note that thermal processing may be performed at different steps in the method, for instance, for obtaining the polymer compound and for further processing of the compound.

The biopolymer may include one or more excipients. Suitable excipients may be used in the invention include one or more of a lubricant, colorant, heat stabilizer, flame retardant, blowing agent, UV stabilizer, cross linking agent, filler, additive, and the like or mixtures thereof. Lubricants may be selected from the group comprising octyl sterate, butyl stearate, Si Oil, stearic acid, stearamide, carnauba and mixtures thereof. Colorants may be selected from the group comprising benzidine-yellow, red 2b pigment, alumina hydrates, iron oxide and mixtures thereof. Heat stabilizers may be selected from the group comprising cadmium, barium, zinc and mixtures thereof. Flame retardant may be selected from the group comprising zinc borate, boric acid, chlorinate parafins, agricultural flour, wood flour and mixtures thereof. Blowing agent may be selected from the group comprising azodicarbonamide citric acid, baking soda and mixtures thereof. UV stabilizer may be selected from the group comprising hydroxybenzophenones, piperidines, benzotriazoles, carbon black and mixtures thereof. Cross liking agent may be selected from the group comprising carboxymethyl cellulose, micro crystalline cellulose, starch, citric acid, joncryl® glutaraldehyde, propionic acid, lignin with glycine and mixtures thereof. Filler may be selected from the group comprising ZnO Nanofillers, tapioca, acetyl tributyl, cellulose nanocrystal, banana stem fiber, egg shell, wood fiber, wood floor, silk powder, lignin, zein, calcium carbonate, talc, kaolin, Fledspar, sisal, Hemp Fiber, green coconut fibers, nanoclay and mixtures thereof. Cross liking agent may be selected from the group comprising poly(lactic acid) [PLA], polyhydroxyalkanoate (PHA), polyhydroxybutyrate (PHB), polybutylene adipate terephthalate (PBAT), Polycaprolactone (PCL), Polybutylene succinate (PBS), Biodegradable Polypropylene (PP), Thermoplastic starch (TPS), Polyamide 11(PA11), Polyvinyl alcohol (PVA), Lyocell Fiber and mixtures thereof.

In yet another embodiment, the film may be directly thermally processed with one or more excipients by way of extruding, molding or casting to produce the end product. Preferably, compounding of keratin with one or more plasticizers, one or more additives, and one or more cross-linking agents at an elevated temperature in the presence of external pressure (for e.g. in a kneader and extruder) will substantially decrease the production time of manufacturing biopolymer using keratin protein.

In a preferred embodiment, the present biopolymer can be made by the method as mentioned above. The biopolymer may be obtained in the form of a pellet, a granule, an extruded solid, a blow film sheet, an injection molded solid, thermoforming sheet, laminating sheet, compression molded sheet a hard foam, a sheet, a dough or a corresponding mold article.

In another embodiment, the biopolymer of the present invention may include a wide variety of polymer materials. The said biopolymer may be combined with any of the existing natural polymer, chemically synthesized polymer, microbial polyester or a mixture thereof to enhance physical properties and reduce the cost of production of biopolymers.

### ADVANTAGES OF THE PRESENT INVENTION

1. The method of present invention is economically and commercially feasible and efficient for producing biopolymer from poultry feathers.
2. The addition of additives at the specified amount results into providing better properties such as high tensile strength, resistance, flexibility and durability to the biopolymer.
3. Compounding of keratin with one or more plasticizers, one or more additives, and one or more cross-linking agents at an elevated temperature in the presence of external pressure will substantially decrease the production time of manufacturing biopolymer using keratin protein.
4. With the described method above, an end product will be created which can also act as an additive for existing biopolymers, and will decrease their overall cost of production.

### EXAMPLES

Pre-Treatment of Feather

Chicken feather waste was collected from Venky's Chicken, Kamshet, Pune to perform the present invention. Feathers were washed with CTAB and disinfected & degreased with acetone & petroleum ether and cleaned with water. The wet feathers were air dried in for 2 days, then, the cleaned feathers, which contained both the fibers and quills, were crushed by a stainless steel pulverizer.

Extraction of Feather Keratin (FK)

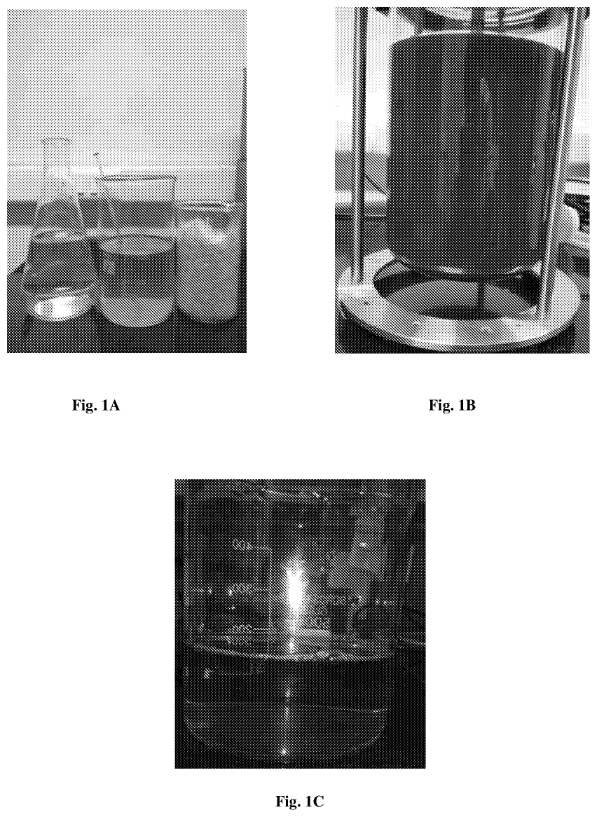

In the hydrolytic process the defatted feathers (10 g) were mixed with Na₂S solutions of various concentrations of 0.1M-0.5M as shown in FIG. 1A, at 60° C. for 6 hours at 70 rpm with a magnetic stir bar to prevent aggregation of the feathers during the reaction.

The prepared hydrolysate, as shown in FIG. 1B, was filtered twice and centrifuged at 10,000 rpm or any machine generating similar condition is used to separate the supernatant, as shown in FIG. 1C. The pH of the solution was adjusted to 3.5 and standardized. The perticipated keratin sediment as shown in FIG. 2A was collected, washed, freeze-dried to obtain microporous keratin particles as shown in FIG. 2B.

Preparation of Keratin Biopolymer Film

Scenario 1

The keratin powder was used to synthesize a biopolymer film using glycerol (1%-3.5%), starch (0.5% -3%) and Carboxymethyl cellulose (0.2%-1%) in NaOH.

To prepare the biopolymer film, 250 mg of dried keratin was dissolved in 5 ml of NaOH (0.5N) under 300 rpm vigorous agitation at 60° C. After Mixing of Keratin proteins with NaOH add glycerol (1-1.5%) with Starch (0.5-3%).

Carboxymethyl Cellulose was gelatinized at a temperature in the range of 60° C. to 100° C. with continuous stirring for 5 min, allowed to cool down and then add with Keratin-Starch mixture. The mixture was poured on petri plate having 10 cm of diameter greased with greasing agent and dried in oven at 60° C. for 48 h.

In this method, firstly the casted keratin blend was cut into granules and was extruded to produce pellets of Feather Keratin Protein (hereinafter referred to as “FKP”)-PBAT. For the production of blends, the blend was obtained by mixing granular FKP, glycerol, and PBAT in a homogenizer.

The neat blend of polymers FKP-PBAT were dried under vacuum at 60° C.-80° C. for 4 hours, before processing and rheological characterizations. In order to avoid hydrolytic degradation Joncryl® was used, which was commercially purchased from BASF. Compositions containing 30, 40, and 50% of casted FKP in weight were studied, against respectively designated PBAT.

All the formulations were melt-compounded in a twin screw extruder with screw diameter of 25 mm and L/D ratio of 22. The mixture is extruded with a temperature profile of 115-135° C. and the screw speed was set at 35 RPM. The cylindrical profiles were cooled to room temperature and cut to obtain the pellets.

Scenario 2

To prepare the biopolymer film, lg of dried keratin was dissolved in 20 ml of NaOH (0.5 N) under 300 rpm vigorous agitation at 65° C. PVA (12.5-40%) was dissolved in DH₂O at 80° C. and add it to Keratin solution to make a mixture and the pH is adjusted to 9 using 0.1N NaOH solution.

Mixture was stirred for 45 min at 65° C., Glycerol was added as a plasticizer and it was stirred for 5 min. After stirring, the mixture was poured on a petriplate having 10 cm of diameter coated with any greasing agent and dried in oven at 70° C. for overnight, as shown in FIG. 2C.

Scenario 3

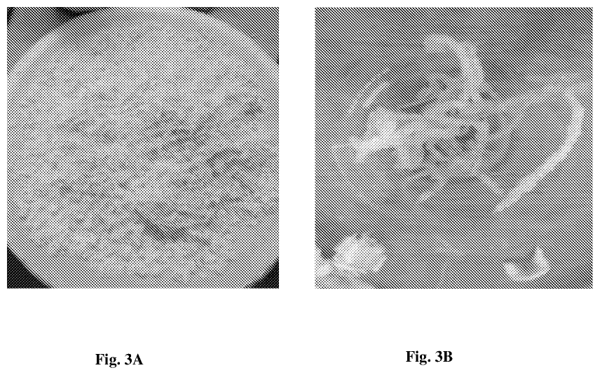

Keratin Protein and other additives were mixed and grinded thoroughly in a two roll mill or a kneader. General composition for protein based biopolymer was FKP (41%), additives (41%), and plasticizer (3-18%). lubricant (0.5%) and filler were added. Various concentrations (0%-60%) of the filler were added by total weight of the respective composition, as shown in FIG. 3A and this mixture was blended using two roll mill or a kneader. Mixture was blended for 20 min at 100° C.-200° C., after blending it was palletized, as shown in FIG. 3B.

### Example-1

Firstly, mixture was blended using Kneader at 100° C.-130° C. and a dough is formed. The crude mixtures of FKP and PBAT is palletized with different compositions and extruded at a temperature of 115 to 130° C. in the four heating zones. Different compositions prepared are 53060, 54050 and 55040 denote 30, 40 and 50 g of Keratin to 100 g of FKP-PBAT, respectively. The cylindrical profiles were cooled to room temperature and cut to obtain the pellets.

The pellets were then pressed at 200° C.- 220° C., using 2-6 ton for 1-2 hour, in a thermopress with hot plate temperature control and the films were subsequently maintained at room temperature. The blown films were prepared in a single screw extruder with 25 mm diameter screw, L/D 22. An annular die of 50 mm diameter, with die gap of 0.85 mm was used to shape the initial tube dimensions. The processing temperature at different zones was set from 130-150° C. for FKP/PBAT blend. The screw speed in the film extruder was set at 30 RPM in formulations with neat PBAT and 35 RPM for FKP-PBAT blends. The take-up speed was set to 2.8 m/min.

### Example-2

The blends of different compositions of FKP-PBAT are prepared with compatibilizers. The processing conditions used are: 30/70, 40/60, 50/50 ratio (FKP/PBAT), addition of maleic anhydride and joncryl® (1-2 and 0.15-0.25% with respect to FKP/PBAT weight, respectively). Firstly, mixture was blended using a Kneader at 100° C.-130° C. and a dough was formed. Then, the mixture was extruded with a temperature profile of 115-130° C., Screw rate of 35 rpm, screw with a compression ratio of 5:1 and L/D ratio of 22 employing a rod die, coupling a 1-mm diameter nozzle at its opening. It was pelletized and vacuum packed.

The pellets were then pressed at 220° C., using 2-4 ton for 1.5 hour, in a thermopress with hot plate temperature control and the films were subsequently maintained at room temperature.

For injection molding, initially, the FKP and PBAT pellets were dried at 80° C. for 6 hours. The granulates were injection moulded with a Ferromatic Milacron injection moulding machine equipped with a screw of 35mm Diameter. Injection molding was performed at a temperature profile of 140° C.-150° C.

Dumbbell specimens were injection moulded for the tensile and shrinkage measurements. The injection moulding pressure was 1000-1400 bars, and the holding pressure was varied from 600 to 1000 bars to determine the effect of the holding pressure on the time dependence of blend. The injection moulded specimens were stored different ways and for different time periods to determine the effect of ageing time and different storage type.

### Example-3

Keratin Protein and other additives were mixed and grinded thoroughly in a two roll mill. Different compositions for protein based biopolymer were FKP (30-50%), PBAT (30-50%), and Glycerol (3-18%), Citric acid (1-1.5%). Steric acid (0.5%) and of the Talc (5%) were added by total weight of the respective composition, as shown in FIG. 3A and this mixture was blended using Kneader.

The compounding of materials in this section was carried out on a thermofisher labscale twin-screw extruder. The material to be compounded was fed at a constant speed into the hopper of the thermofisher labscale extruder by means of a screw feed system. The mixture was extruded at the temperature profile of 120-140° C. Extruder screw speeds in this section were set at 35 RPM to produce composites containing 20 wt. % of fillers, additives, stabilizers, & cross linking agents.

The blown films were prepared in a single screw extruder with 25 mm diameter screw, L/D 22. An annular die of 50 mm diameter, with die gap of 0.85 mm was used to shape the initial tube dimensions. The processing temperature at different zones was set from 135-145° C. for FKP/PBAT blend. The screw speed in the film extruder was set at 30-40 RPM for blends. The take-up speed was set to 2.8 m/min. The various steps of the present invention were performed at a particular temperature profile as described in Table 1.

TABLE 1
Steps of the invention performed within
a particular range of temperature

| Steps | Temperature Range |
|---|---|
| Gelatinization of keratin-starch mixture | 60-100° C. |
| Drying of polymer blend FKP-PBAT | 60-80° C. |
| Kneader temperature | 100-130° C. |
| Temperature profile of extruder for | 115-135° C. |
| casted keratin and PBAT blend | |
| Temperature profile of extruder for | 115-130° C. |
| FKP-PBAT blend | |
| Temperature profile of extruder for | 120-140° C. |
| FKP-PBAT blend with additives | |
| Blow-film temperature | 130-150° C. |
| Injection molding | 140-150° C. |
| Thermopress temperature | 200-220° C. |

Evaluation of Mechanical and Thermal Properties of the Product

The tensile strength of the product was evaluated by a Universal Testing Machine (UTM), in which the test specimens were prepared in a dumbbell shape. The mechanical properties such as tension and elongation, and thermal properties of the flexible Dumbles were evaluated and presented in Table 2. The mechanical properties of the FKP +PBAT films were determined in accordance to the ASTM standard. With the information on the equipment's load and displacement, maximum resistance (MPa), deformation (%) were obtained, to determine changes due to the concentration of the raw FKP-PBAT blend and FKP-PBAT with additives.

TABLE 2
Results of testing the mechanical and
thermal properties of the biopolymer

| | | Tensile | Elongation |
|---|---|---|---|
| | | strength | at break |
| Polymer | % FKP | (MPa) | (%) |
| PBAT | | 21 | 680 |
| Casted FKP- | 30% | 14.8 | 370 |
| PBAT | 40% | 13.4 | 325 |
| | 50% | 10.5 | 265 |
| FKP-PBAT | 30% | 15.7 | <400 |
| blend | 40% | 13.1 | 365 |
| | 50% | 11.4 | 324 |
| FKP-PBAT with | 30% | 16.6 | 304 |
| fillers and | 40% | 14.4 | 259 |
| additives | 50% | 12.8 | 234 |

1. A method of making biopolymer from feathers comprising the steps
i) pre-treatment of native feathers;
ii) extraction of keratin protein from pre-treated feathers in the presence of reducing agent;
iii) polymerization by blending keratin protein with one or more plasticizer, one or more additive and one or more cross-linking agent, optionally in presence of at least one alkali hydroxide, to obtain a polymer compound using one or more thermal processing techniques at a temperature in the range of 60° C. to 150° C.; and
iv) applying pressure and subjecting the polymer compound to thermal processing at a temperature in the range of 100° C. to 220° C. in presence of at least one or more excipients.

2. The method as claimed in claim 1, wherein the pre-treatment step comprises washing with water followed by exposure to various chemicals agents selected from a group comprising SDS, CTAB, Detergent, petroleum ether, acetone, and mixtures thereof.

3. The method as claimed in claim 1, wherein the keratin is obtained from a group comprising feather fiber keratin, feather quill keratin, and mixtures thereof.

4. The method as claimed in claim 1, wherein the reducing agent is selected from a group comprising potassium cyanide thioglycolic acid, sodium sulphide, 2-mercaptoethanol, dithiothreitol, sodium m-bisulphite, and sodium bisulphite, sodium hydroxide, urea, thiourea, and mixtures thereof.

5. The method as claimed in claim 1, wherein the plasticizer is selected from a group comprising glycerol, sorbitol, ethanolamine, formamide, ethylene bisformide, glycerol triacetate, dibutyl tartrate, and mixtures thereof.

6. The method as claimed in claim 1, wherein the additive is selected from a group comprising poly(lactic acid) [PLA], polyhydroxyalkanoate (PHA), polyhydroxybutyrate (PHB), polybutylene adipate terephthalate (PBAT), Polycaprolactone (PCL), Polybutylene succinate (PBS), Biodegradable Polypropylene (PP), Thermoplastic starch (TPS), Polyamide 11(PA11), Polyvinyl alcohol (PVA), Lyocell Fiber, and mixtures thereof.

7. The method as claimed in claim 1, wherein the cross-linking agent is selected from a group comprising carboxymethyl cellulose, micro crystalline cellulose, starch, citric acid, Joncryl®, glutaraldehyde, propionic acid, lignin with glycine, and mixtures thereof.

8. The method as claimed in claim 1, wherein the alkali hydroxide is selected from a group comprising Lithium hydroxide (LiOH), Sodium hydroxide (NaOH), Potassium hydroxide (KOH), Rubidium hydroxide (RbOH), Caesium hydroxide (CsOH), and mixtures thereof.

9. The method as claimed in claim 1, wherein the amount of plasticizer is in the range of 0 wt % to 30 wt %, additive is in the range of 0 wt % to 60 wt %, cross-linking agent is in the range of 0 wt % to 15 wt % and alkali hydroxide is in the range of 0 wt % to 8wt % of the total weight of the biopolymer synthesised.

10. The method as claimed in claim 1, wherein said keratin protein is present in the range of 0 wt % to 90% wt %, preferably between 30 wt % to 60 wt % of the total weight of the biopolymer synthesised.

11. The method as claimed in claim 1, wherein the polymerisation step is conducted at a temperature in the range of 30° C. to 200° C., preferably at 60° C. to 180° C.

12. The method as claimed in claim 1, wherein the polymer compound is processed in the presence of one or more excipients comprising lubricant, colorant, heat stabilizer, flame retardant, blowing agent, UV stabilizer, cross linking agent, filler, additive, and mixtures thereof.

13. The method as claimed in claim 1, wherein thermal processing comprises subjecting the polymer to one or more steps of kneading, extrusion molding, injection molding, blow molding, compression molding, transfer molding, thermoforming, casting, calendering, low-pressure molding, high-pressure laminating, reaction injection molding, foam molding, or coating.

14. A biopolymer prepared using the method as claimed in claim 1.

15. The biopolymer as claimed in claim 14, wherein the biopolymer is obtained in the form of a pellet, a granule, an extruded solid, a blow film sheet, an injection molded solid, thermoforming sheet, laminating sheet, compression molded sheet a hard foam, a sheet, a dough or a corresponding mold article.

16. The biopolymer as claimed in claim 14, further comprising the combination of biopolymer with any of the existing natural polymer, chemically synthesized polymer, microbial polyester, or a mixture thereof.
